(19) United States
(12) Patent Application Publication
(10) Pub. No.: US 2021/0109110 A1
(43) Pub. Date: Apr. 15, 2021

(54) PATIENT ASSESSMENT METHOD

(71) Applicants: ViroGates A/S, Birkerod (DK); Hvidovre Hospital, Hvidovre (DK); University of Copenhagen, Copenhagen K (DK)

(72) Inventors: Jesper Eugen-Olsen, Klempenborg (DK); Ove Anderson, Hvidovre (DK)

(21) Appl. No.: 16/971,891

(22) PCT Filed: Feb. 20, 2019
(86) PCT No.: PCT/EP2019/054232

§ 371 Date: Aug. 21, 2020

(30) Foreign Application Priority Data
Feb. 21, 2018 (GB) 1802795.3

(51) Int. Cl. G01N 33/68; G16H 50/30

(52) U.S. Cl. CPC G01N 33/6872; G16H 50/30; G01N 2800/04; G01N 2800/7095; G01N 2800/52

(57) ABSTRACT

A subject's level of soluble urokinase type plasminogen activator (suPAR) is checked as part of a risk stratification procedure in a hospital emergency department to help decide whether to admit the subject to the hospital, keep the subject in as a patient, or discharge a patient.

### BACKGROUND AND FIELD OF THE INVENTION

The invention concerns the examination of subjects admitted to, or presenting at, a hospital emergency department (hereinafter “ED”, also named Acute Care Department, or Accident & Emergency Department).

Rapid and safe risk stratification is a necessary and important task in emergency medicine. “Risk stratification” in this context means classifying patients into bands or groups according to the perceived risk of their needing in-hospital care. Identifying subjects at high and low risk shortly after admission can guide clinical decision-making towards the patients in need, regarding treatment, observation and allocation of resources and those not in need of a hospital admission. Several studies have suggested biomarkers as a supplement to enhance risk stratification; however, they have only been studied retrospectively, which is why an interventional study was both warranted and required, in order to quantify the effects of implementing a prognostic biomarker in emergency medicine. The current invention results from a study that was to our knowledge the first of its kind. The study focused on whether the availability of a prognostic biomarker influences the treatment strategy and overall prognosis of subjects admitted to the ED.

A biomarker reflecting the level of urgency or comorbidity (two or more co-existing diseases) burden is potentially very useful, but the value of a biomarker with a strong negative predictive value must not be underestimated. The availability of a biomarker reflecting healthiness or non-urgency is particularly interesting in the setting of emergency departments where crowding is a serious concern. High bed occupancy rates are associated with an increased mortality (i.e. death) rate, delays in initiation of time-critical care and diagnosis, increased costs and an overall poor quality of care and concerns of patient safety. Furthermore, hospitalization is associated with a number of adverse outcomes such as falls, medication errors, in-hospital infections, and delirium. Early discharge is associated with decreased mortality and increased patient outcome, illustrated by an American study and a British study that found 26% or 20%, respectively, of all hospitalizations were potentially avoidable. A more efficient identification of subjects who do not need to be admitted is desirable.

The present invention aims to provide a novel means by which medical personnel can (in conjunction with other clinical observations and medical history etc) assess the state of a subject and, in particular, the subject's risk of mortality within a short time frame. This enables more accurate assessments to be made concerning whether a subject should be admitted or discharged.

### PRIOR ART

WO 2008/077958 (Hvidovre Hospital) discloses the use of soluble urokinase-type plasminogen activator receptor (suPAR) as a biomarker for low-grade inflammation (LGI), diseases associated with LGI, and metabolic syndrome. It also discloses the measurement of suPAR levels in apparently healthy subjects as a means of assessing the risk of developing a disease (such as cardiovascular disease) and the overall risk of mortality within ten years, principally so that lifestyle changes can be made in order to reduce those risks. Determining the risk of developing a disease (as opposed to having the disease) and the risk of mortality within ten years in an apparently healthy subject is not relevant to the sort of assessments that are needed in an ED.

Rasmussen et al (2016) Emerg. Med. J. 0, 1-7 discloses the use of suPAR levels as a prognostic marker in patients admitted to an ED. It was a retrospective study and the results were equivocal. For example, the authors concluded that “the association we found between high suPAR and readmission at the time of admission may not be clinically applicable per se, but support that suPAR is a surrogate marker of disease severity or additional underlying disease and could raise awareness of morbidity other than the acute illness already from the point of admission”.

Similar equivocal disclosures are to be found in Østervig et al (2015) Sc. J. Trauma, Resusc. and Emerg. Med. 23 (Suppl 1) A31; Haupt et al (2012) Critical Care 16, R130; Nayak et al (2015) Dan. Med. J. 62, A5146; and on the ClinicalTrials.gov website ref NCT02643459.

Accordingly, a clinical trial was devised in order to determine whether measuring suPAR levels would be useful in deciding whether to admit, keep in, or discharge a subject in an ED. The design of the trial has been published in Sandø et al (2016) Sc. J. Trauma, Resusc. and Emerg. Med. 24, 100-106 but the results have not yet been published. Thus, according to the state of the art, it is not currently known whether the suPAR measurements are useful in this context. The present invention is based on (unpublished) results showing that the suPAR measurements are useful in this context.

### SUMMARY OF THE INVENTION

One aspect of the invention provides a method of applying risk stratification to a human subject who has been admitted to, or presents at, a hospital emergency department (ED), the method comprising measuring the subject's suPAR level and comparing it with a reference value.

The risk stratification may comprise triaging the subject, determining the ED-relevant health status of the subject, improving the disease risk identification in acute medical patients, identifying whether serious disease is present or not at time of presentation in the ED, and/or providing support for the clinical decision of discharge or admittance of the acute medical patient.

The triaging method may comprise determining the morbidity of the subject (including risk of in-hospital death), or the risk of death within 28 days, 30 days, 90 days or 6, 10 or 12 months of the subject, or the need to admit the subject into the hospital, or the ability to discharge the patient from the hospital. “Morbidity” is the state or extent of being diseased.

The measurement of the suPAR level is typically carried out in vitro on a sample taken from the subject. The sample is typically blood, blood serum, blood plasma, cerebrospinal fluid or urine. The sample may undergo processing before the measurement is carried out. For example, it might be centrifuged, frozen and thawed, diluted, concentrated, stabilised, filtered, dried onto filter paper or treated with preservative.

### DETAILED DESCRIPTION OF THE INVENTION

Urokinase-type Plasminogen Activator Receptor (uPAR, CD87) is the cellular receptor for urokinase (uPA), and is expressed by most leukocytes, including monocytes, macrophages, neutrophils and platelets. uPAR is an activation antigen in monocytes and T cells. uPAR may be shed from the cell surface, generating a soluble form of the receptor (suPAR) lacking the GPI-anchor. The shedding mechanism is poorly understood but may occur by cleavage of the GPI-anchor catalyzed by a GPI-specific phospholipase D. Soluble forms of uPAR (suPAR) have been identified in cell culture supernatants and in diverse biological fluids such as tumor ascites, cystic fluid, serum, cerebrospinal fluid, plasma and urine. The cellular origin of circulating suPAR is not known. Many, if not all, cells which express uPAR also shed soluble forms of the receptor when cultured in vitro.

The protein suPAR (NCBI Accession no. AAK31795 and isoforms of the receptor, NP_002650, 003405, NP_002650, NP_OO1005376) is the soluble portion of Urokinase-type Plasminogen Activator Receptor (uPAR), which is released by cleavage of the GPI anchor of membrane-bound uPAR. suPAR is a family of glycosylated proteins consisting of full length suPAR (277 amino acids (1-277)) and suPAR fragments D1 (1-83), and D2D3 (84-277) generated by urokinase cleavage or human airway trypsin-like protease, D1 (1-87) and D2D3 (88-277) generated by MMP cleavage, D1 (1-89) and D2D3 (90-277) also generated by urokinase cleavage or human airway trypsin-like protease, D1 (1-91) and D2D3 (92-277) generated by cleavage by plasmin. Continuous and discontinuous epitopes present in the protein suPAR and its cleavage products may be used to monitor their presence and abundance in a biological fluid by immunodetection with mono- or polyclonal antibodies. Antibodies directed to accessible epitopes common to suPAR and its cleavage products (e.g. D2D3) can be used to detect both suPAR and its cleavage products in a biological fluid. Since there is a one-to-one relationship between suPAR and its cleavage products, an antibody that is directed to an epitope that is common to both full length suPAR and, say, the D2D3 cleavage product will at the same time directly and indirectly measure the suPAR level. That is to say, a value of, say, 3 ng/ml as measured in the assay is regarded as indicating a suPAR level of 3 ng/ml, even though some of the protein that was detected may have been the D2D3 cleavage product. In the context of the assay, therefore, “suPAR” refers to full length suPAR and its cleavage product D2D3. The term D2D3 is used to denote any suPAR-derived fragment corresponding to the 84-277 region of suPAR and having an N-terminus lying in the 84-92 amino acid region of suPAR and a C-terminus corresponding to the C-terminus of suPAR (amino acid 277), for example 84-277, 88-277, 90-277 and 92-277.

suPAR is a broadly applicable prognostic biomarker with potential use in a broad variety of acute and chronic diseases, and it is also a predictor of long term disease development in the general population. It was known that suPAR is an unspecific biomarker with prognostic value across various diseases but we now show for the first time that it is a useful biomarker for risk stratification in an ED, as the staff can target intervention, resources, and clinical focus where most beneficial and, through this knowledge and intervention, reduce mortality.

When a subject presents at the Emergency Department (ED) with an acute medical condition, vital signs, scoring systems and a range of biomarkers are used in a triage process to determine the urgency of the subject's needs and to diagnose and prognosticate the subject. A range of biomarkers including soluble urokinase plasminogen activator receptor (suPAR) have shown prognostic value in retrospective studies. The suPAR biomarkers reflect the severity and prognosis of the subject, but until the present invention it was unknown whether this knowledge, in addition to the knowledge already available to the physician, could alter the outcome of the subjects. Outcomes can be defined as morbidity, admissions, readmissions or mortality (following discharge from hospital or in-hospital mortality) within a specified period, for example 1, 2, 3, 4, 5, 6, 7, 8, 9, 10, 11 or 12 months with reference to those with a high level of suPAR or number of patients discharged within 24 hours or mean length of stay in hospital, with reference to the use of low values of suPAR (negative predictive value).

Outcome can also be related to the negative predictive value of suPAR, e.g. low suPAR resulting in quick discharge, shorter length of stay. In other words, the methods of the invention can be used in identifying those with a low risk of disease, thereby improving patient flow in the hospital, and reducing the number of unnecessary admissions, and thereby also lead to a shortening of length of stay.

This can also be seen in the light of a significant effect of measuring suPAR in the TRIAGE III trial with regard to reducing number of patients admitted to hospital, and shortening their length of stay, even if there is no effect on overall mortality.

The risk stratification method of the invention can additionally measure and/or process one or more of: the subject's sex, age, medical history, haemoglobin level, C Reactive Protein level, creatinine level, leucocyte count, sodium level, potassium level, adrenomedullin level, albumin level, D-dimer level, troponin level (HEART Score); recording clinical symptoms and signs such as physiological parameters, such as pulse, cognition, blood pressure, temperature and respiratory rate; the output of a risk algorithm such as Early warning score and similar and locally adapted variables thereof (e.g. Decision-tree early warning score (DTEWS) or National Early Warning Score (NEWS), Acute Physiology and Chronic Health Evaluation (APACHE), Glasgow coma scale, electrocardiogram, age, risk factors, quick Sepsis Related Organ Failure Assessment (qSOFA), or the Model for Endstage Liver Disease (MELD), based on bilirubin, INR (international normalized ratio), and creatinine). An account of the Early Warning Score, for example, can be found in Alam et al (2014) Resuscitation 85, 587-594. Further examples include the American Society of Anesthesiologists (ASA) classification (which is a simple six-point scale used in the preoperative setting, used to assess the surgical patients' overall physical status); the Physiologic and Operative Severity Score for the enUmeration of Mortality and Morbidity (POSSUM) score; and other risk scores for outcome prediction of acute hospitalized patients, such as the GRACE ACS Risk and Mortality Calculator (which estimates admission-6 month mortality for patients with acute coronary syndrome), the Thrombolysis in Myocardial Infarction risk score (TIMI RS), Platelet glycoprotein IIb/IIIa in Unstable angina: Receptor Suppression Using Integrilin Therapy risk score (PURSUIT RS), and Global Registry of Acute Cardiac Events risk score (GRACE RS) for in-hospital and 1 year mortality across the broad spectrum of non-ST-elevation acute coronary syndromes (ACS).

A further aspect of the invention provides apparatus for applying risk stratification to a human subject who has been admitted to, or presents at, a hospital emergency department (ED), the apparatus comprising:
means to accommodate a sample obtained from the subject,
a detector configured to measure the level of soluble urokinase type plasminogen activator (suPAR) in the sample,
a processing module to compare the level of suPAR with a reference suPAR value, and
means to output a risk stratification.

The means to output the risk stratification may be a visual display or a printout.

In order to output the risk stratification, the apparatus may additionally measure and/or process one or more of: the subject's sex, age, medical history, haemoglobin level, C Reactive Protein level, creatinine level, leucocyte count, sodium level, potassium level, adrenomedullin level, albumin level, D-dimer level, troponin level (HEART Score); recording clinical symptoms and signs such as physiological parameters, such as pulse, cognition, blood pressure, temperature and respiratory rate; the output of a risk algorithm such as Early warning score and similar and locally adapted variables thereof (e.g. Decision-tree early warning score (DTEWS) or National Early Warning Score (NEWS), Acute Physiology and Chronic Health Evaluation (APACHE), Glasgow coma scale, electrocardiogram, age, risk factors, quick Sepsis Related Organ Failure Assessment (qSOFA), or the Model for Endstage Liver Disease (MELD), based on bilirubin, INR (international normalized ratio), and creatinine). An account of the Early Warning Score, for example, can be found in Alam et al (2014) Resuscitation 85, 587-594. Further examples include the American Society of Anesthesiologists (ASA) classification (which is a simple six-point scale used in the preoperative setting, used to assess the surgical patients' overall physical status); the Physiologic and Operative Severity Score for the enUmeration of Mortality and Morbidity (POSSUM) score; and other risk scores for outcome prediction of acute hospitalized patients, such as the GRACE ACS Risk and Mortality Calculator (which estimates admission-6 month mortality for patients with acute coronary syndrome), the Thrombolysis in Myocardial Infarction risk score (TIMI RS), Platelet glycoprotein IIb/IIIa in Unstable angina: Receptor Suppression Using Integrilin Therapy risk score (PURSUIT RS), and Global Registry of Acute Cardiac Events risk score (GRACE RS) for in-hospital and 1 year mortality across the broad spectrum of non-ST-elevation acute coronary syndromes (ACS).

Biological Samples Suitable for Detection of suPAR as a Marker

suPAR and its cleavage products (e.g., D2D3) can be used as a marker for the purposes of the invention by measuring the level of suPAR in a biological fluid derived from a human subject, as illustrated in the examples herein. suPAR and its cleavage products are present in all biological fluids derived from a human subject, including cerebrospinal fluid, plasma, serum, blood, urine, semen, saliva and sputum.

Preferably, the sample is plasma or serum.

Where the biological sample is urine, the measurements may be based on the urine suPAR/creatinine value from a subject, since this value is known to be highly correlated to the concentration of suPAR in a plasma sample derived from the same subject. Thus, urine samples may also be employed for the measurement of suPAR, where the measured level in urine is normalized for protein content (e.g. using creatinine). These normalized values may be employed as a marker for the purposes of the present invention.

Detection and Quantitation of suPAR and its Cleavage Products

Accurate methods for measuring the level of suPAR in a biological fluid derived from a subject include immunodetection methods, e.g. Enzyme-Linked ImmunoSorbent Assay (ELISA), which are particularly suitable as such methods are relatively cheap and simple to perform in the clinical setting. ELISAs can be adapted to analyze both small and large numbers of samples, and include both an ELISA plate format with wells coated with suPAR specific antibodies, or adapted to a lateral flow format incorporating components of the ELISA assay. Additionally, suPAR levels can be measured by proteomic approaches such as western blot, Luminex, MALDI-TOF, HPLC and automated immune analyzer platforms such as Bayer Centaur, Abbott Architect, Abbott AxSym, Roche COBAS and the Axis Shield Afinion. A suitable ELISA or lateral flow device, suPARnostic® quick test or turbidimetric assay suPARnostic® Turb are available commercially from Virogates A/S, Birkerød, Denmark, under the trade name suPARnostic®.

Monoclonal antibodies to the said receptor or receptor peptides used in the method of the present invention may be prepared using any technique which provides for the production of antibody molecules by continuous cell lines in culture. These include, but are not limited to, the hybridoma technique, the human B-cell hybridoma technique, and the EBV-hybridoma technique. See, e.g., Kohler, et al, 1975, Nature 256: 495-497; Kozbor et al, 1985, J. Immunol. Methods 81: 31-42; Cote et al, 1983, Proc. Natl. Acad. Sci. USA 80: 2026-2030; Cole et al, 1984, Mol. Cell Biol. 62: 109-120. Specifically, the method comprises the following steps: (a) immunizing an animal with an immunogenic receptor peptide; (b) isolating antibody producing cells from the animal; (c) fusing the antibody producing cells with immortalized cells in culture to form monoclonal antibody-producing hybridoma cells; (d) culturing the hybridoma cells; and (e) isolating from the culture monoclonal antibodies which bind to said polypeptide.

Antigenic specificity is conferred by variable domains and is independent of the constant domains, as is known from experiments involving the bacterial expression of antibody fragments, all containing one or more variable domains. These molecules include Fab-like molecules (Better et al (1988) Science 240, 1041); Fv molecules (Skerra et al (1988) Science 240, 1038); single-chain Fv (ScFv) molecules where the V_{H} and V_{L} partner domains are linked via a flexible oligopeptide (Bird et al (1988) Science 242, 423; Huston et al (1988) Proc. Natl. Acad. Sci. USA 85, 5879) and single domain antibodies (dAbs) comprising isolated V domains (Ward et al (1989) Nature 341, 544). A general review of the techniques involved in the synthesis of antibody fragments which retain their specific binding sites is to be found in Winter & Milstein (1991) Nature 349, 293-299. By “ScFv molecules” we mean molecules wherein the V_{H} and V_{L} partner domains are linked via a flexible oligopeptide. These molecules may be used in the present invention.

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between the polypeptide(s) of the present invention and its specific antibody.

The reference value with which the subject's suPAR level is compared is typically 0-16 ng/ml in terms of the plasma level. The test can be applied to whole blood, in which case there will be a barrier to hold back the red blood cells, such that the test effectively measures the level in plasma.

Today, there are many patients that are admitted to hospital that, with the knowledge of suPAR, could be discharged without increasing risk of readmittance or mortality. It is the patients with a suPAR level of lower than 4 ng/ml, especially lower than 3 ng/ml, that need not be admitted as a patient, and can be discharged from the hospital.

In patients that have suPAR above 3 ng/ml and especially above 4 ng/ml, but below 6 ng/ml, the suPAR level is an indicator of the presence of disease and supports the doctor in acknowledging that the patient is diseased.

A suPAR level of higher than 6 ng/ml is a strong factor indicating that a subject should be admitted as a patient, or kept in as a patient, even if other components of the risk stratification procedure are factors indicating that the subject need not be admitted or can be discharged. That is to say, it is likely that a decision will be made to admit the subject as a patient, or to keep them in as a patient, even if there is no other factor indicating that this should be done.

A suPAR level above 9 ng/ml is a strong factor that the patient is of risk of mortality and should be admitted and given a high level of clinical attention, even if other parameters suggest that the patient could be discharged.

Preferably, the subject's suPAR level is measured within 1, 2, 3, 4, 5 or 6 hours of the subject's arrival at the hospital emergency department or even in the ambulance before arrival at the hospital.

### FIGURES

FIG. 1 shows linear correlation between fasting plasma suPAR versus overnight fasting urine suPAR corrected for urine creatinine in a sub-sample of 24 HIV-infected patients, where both scales are log transformed. The strength of the correlation is given as R².

FIG. 2 shows a pocket assessment card to be used by medical staff when making use of the method of the invention. It illustrates suPAR level interpretation and mortality risk stratified by suPAR intervals. ED—emergency department, suPAR=soluble urokinase plasminogen activator receptor, COPD=chronic obstructive pulmonary disease.

FIG. 3 shows the flow-diagram of the included patients.

FIG. 4 shows the number of patients discharged within 24 hours in the group with a suPAR measurement and the controls.

FIG. 5 shows the length of hospital stay in patients with suPAR measured at inclusion and patients without (controls).

FIG. 6 is a ROC curve analysis for single markers and their ability to predict 30-day mortality.

FIG. 7 is a suPAR patient-flow guideline from the TRIAGE III study.

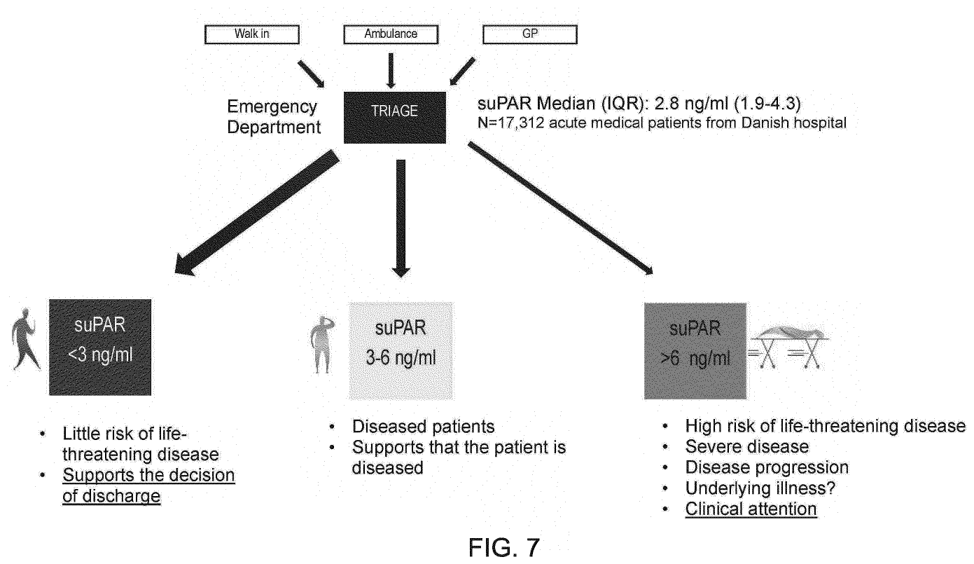

FIG. 8 shows how the addition of a suPAR measurement and comparison with a reference value increases the specificity and sensitivity of a 30 day mortality assessment.

FIG. 9 shows how the addition of a suPAR measurement and comparison with a reference value increases the specificity and sensitivity of a 90 day mortality assessment.

### EXAMPLE 1—MEASUREMENT OF SUPAR LEVEL

suPAR levels may be measured in body fluids by the methods taught in WO 2008/077958, which is incorporated herein for that purpose.

More specifically, suPAR levels may be determined by ELISA assay as follows: Nunc Maxisorp ELISA-plates (Nunc, Roskilde, Denmark) are coated overnight at 4° C. with a monoclonal rat anti-suPAR antibody (VG-1, ViroGates NS, Copenhagen, Denmark, 3 μg/ml, 100 μl/well). Plates are blocked with PBS buffer+1% BSA and 0.1% Tween 20, 1 hour at room temperature, and washed 3 times with PBS buffer containing 0.1% Tween 20. 85 μl dilution buffer (100 mm phosphate, 97.5 mm NaCl, 10 g L⁻¹ bovine serum albumin (BSA, Fraction V, Roche Diagnostics GmbH Penzberg, Germany), 50 U mL⁻¹ heparin sodium salt (Sigma Chemical Co., St. Louis, Mo.), 0.1% (v/v) Tween 20, pH 7.4) containing 1.5 μg/ml HRP labeled mouse anti-suPAR antibody (VG-2-HRP, ViroGates) and 15 μl plasma (or serum or urine) sample is added in duplicates to the ELISA plate. After 1 hour of incubation at 37° C., plates are washed 10 times with PBS buffer+0.1% Tween 20 and 100 μl/well HRP substrate added (Substrate Reagent Pack, R&D Systems Minneapolis, Minn.). The colour reaction is stopped after 30 min using 50 μl per well 1M H₂S0₄ and measured at 450 nm.

Furthermore, suPAR can be measured in bodily fluids using commercially available CE/IVD approved assays such as the suPARnostic product line according to the manufacturer's instructions. In the TRIAGE III trials, suPAR was quantified using the suPARnostic Quick Triage lateral flow assay.

### EXAMPLE 2—CORRELATION OF PLASMA AND URINE LEVELS OF SUPAR

WO 2008/077958 shows that plasma levels of suPAR in HIV-infected patients on stable HAART correlate with urine suPAR, as has been demonstrated previously in HIV negative individuals, and that diurnal changes in urine suPAR are small (Sier et al., 1999, Lab Invest 79:717-722). A sub-sample of 24 of 36 patients had provided overnight-fasting urine. The effect of differences in dilution of the urine on suPAR levels was corrected with the amount of creatinine, as described previously (Sier et al, 1999, Lab Invest. 79:717-722). Urine creatinine was measured as described (Mustjoki et al, 2000, Cancer Res. 60:7126-7132).

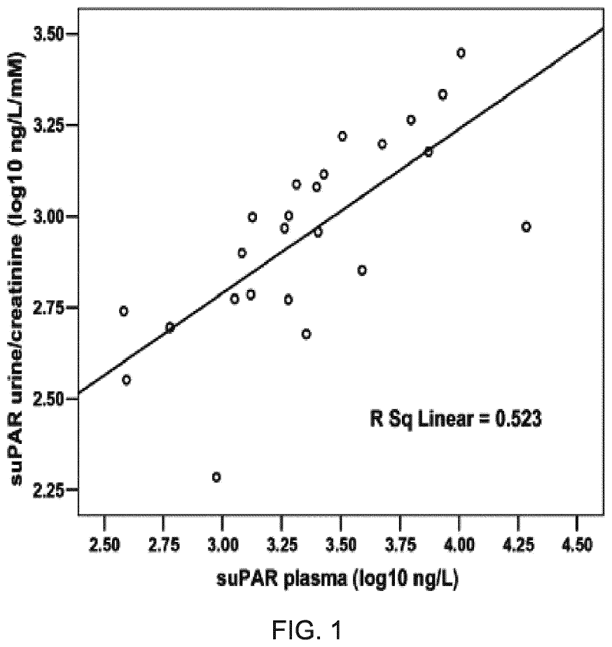

FIG. 1 shows that fasting plasma suPAR and urine suPAR are highly correlated in HIV-infected patients on stable HAART. Since urine suPAR is shown to be a robust estimate of plasma suPAR, the level of suPAR can be performed on urine as well as plasma samples from such individuals. There is no reason to suppose that a similar correlation, and an equivalent correction factor, cannot be used in all subjects.

### EXAMPLE 3—CLINICAL TRIAL STRUCTURE

A randomized intervention study was carried out at two large hospitals in the capital region of Denmark (ClinicalTrials.gov number, NCT02643459). The hypothesis of the study was that the introduction, fast measurement and immediate reporting (knowledge) of the suPAR level to attending physicians or other hospital professionals in the EDs will be associated with a reduction in all-cause mortality at least 10 months after admission.

The primary aim of the study was to evaluate whether the determination of the subject's suPAR level can be used as a part of risk stratification of unselected acutely admitted subjects in order to reduce all-cause mortality.

The secondary aims included:
All cause mortality after index admission, after 30 days.
Number of discharges from the emergency room within 24 hours.
Length of stay during admission. [Time Frame: In-hospital stay].
Number of readmissions [Time Frame: 30 and 90 days]. All new admissions within 91 days of the same patient are defined as readmissions.
Economical expenses [Time Frame: in-hospital stay, 30 days and 10 months after inclusion period ends].

The main hypothesis was to assess if all-cause mortality at 10 months after admission is lower when the suPAR biomarker is measured on acutely admitted patients. Using a 5% level of significance and a power of 80%, a sample of 7340 subjects was needed in each randomization group to detect an absolute risk reduction in mortality at least 10 months after admission of 1.5%.

TABLE 1
Trial structure

| Cycle | 1 | 2 | 3 | 4 | 5 | 6 |
|---|---|---|---|---|---|---|
| Hospital 1 | +suPAR | Control | +suPAR | Control | +suPAR | Control |
| Hospital 2 | Control | +suPAR | Control | +suPAR | Control | +suPAR |

Each cycle consisted of three weeks with (+suPAR) or without (Control) suPAR measurements in the ED.

Quantification of suPAR

Blood samples (6 mL EDTA plasma tubes) for measurement of plasma suPAR were drawn along with the routine blood work. For quantification of suPAR, blood collection tubes were spun for 60 s at 6000 RPM. 10 μL of plasma was added to a prefabricated tube containing 100 μL of running buffer. Using a 60 μL pipette, the plasma and buffer were mixed by pipetting the solution up and down 5 times. From this mixture, 60 μL was added to the suPARnostic® Quick Triage stick, a lateral flow device (also called suPARnostic® Quick Test). After 20 min, the lateral flow device was visually inspected for test and control line, and the suPAR test line quantified using a suPARnostic Quick test device reader (Qiagen, Germany) [20]. According to the test manufacturer (ViroGates NS, Birkeroed, Denmark), the limit of Detection (LOD) for the suPARnostic quick test was 0.3 ng/ml. The limit of quantification (LOQ) was 2 ng/mL defined at the lowest concentration with a CV % that does not exceed 25%. The intra- and interserial measured CV % on 5 samples×4 concentrations (2.0; 4.0; 8.4; 13.7 ng/mL) measured on the same day or with 5 days interval was less than 25%. The r² of the suPARnostic Quick Test compared to the suPARnostic ELISA is 0.875. Analysis of suPAR level was handled by trained medical students according to the manufacturer's instructions, available on-site full-time for non-stop inclusion of eligible subjects. All suPAR levels were analyzed as quickly as possible and always within two hours following blood sampling and immediately reported.

### Information to Physicians

The suPAR level was presented to the attending physicians through the electronic systems LABKA, OPUS and Cetrea. LABKA II (v. 2.5.0.H2, Computer Sciences Corporation (CSC)) is the clinical laboratory information system used to request blood work and view results from laboratory analysis. OPUS (OPUS Arbejdsplads, v. 2.5.0.0, Computer Sciences Corporation (CSC)) is the electronic database of medical records. The emergency wards in the EDs are monitored by the Cetrea system, which is presented by several large screen monitors in the ED and presents a rough overview of the ward (patient data and status, possible diagnosis, route of admission) used by physicians and nurses. Prior to the study, all physicians working in the emergency department were informed in writing about the prognostic abilities of suPAR in unselected subjects, and in regard to specific diagnoses in the form of a review of published literature, as well as pocket cards providing unadjusted mortality rates from 10,000 subjects from similar EDs.

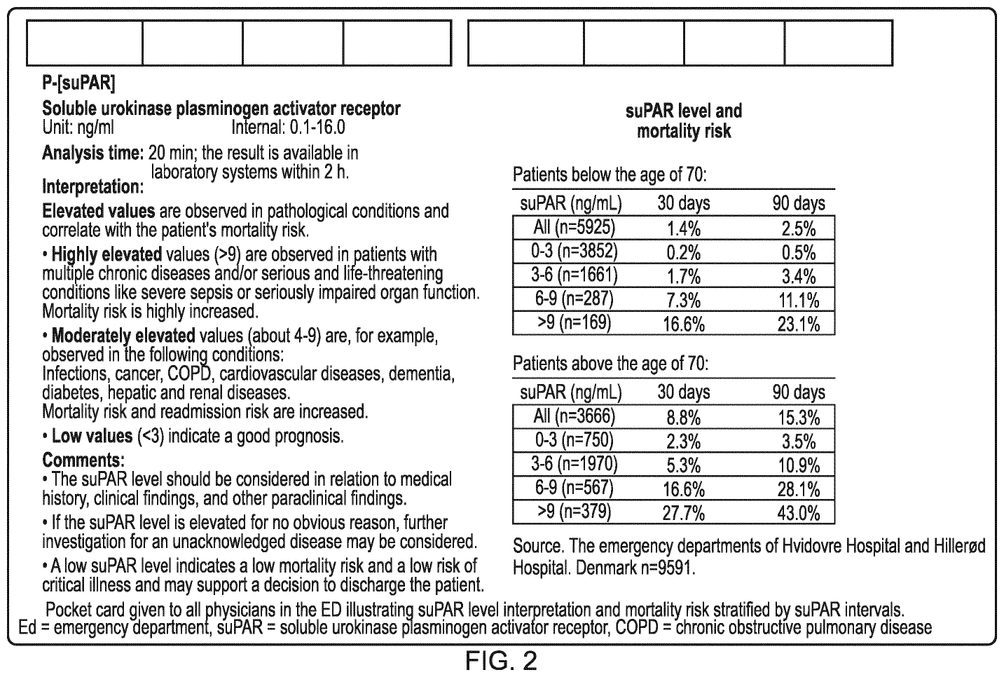

The participating doctors and nurses were informed that they should consider the high risk connected with increased suPAR levels, and clinical reconsideration was advised when encountering a subject with an unexplained high suPAR, in which case an individual intervention should be scheduled based on symptoms and objective findings for the particular clinical issue, for example referral to a specialist, follow-up consultation with general practitioner, positron emission tomography scan or other diagnostic procedures or scanning methods. On the other hand, a low suPAR should promote faster discharge. The doctors were informed of specific cut-of values with regard to suPAR and age and the mortality risk associated with those values (FIG. 2). The data in these information charts was based on retrospective patient data obtained from North Zeeland and Copenhagen University Hospital Hvidovre, Denmark.

For the sake of clarity, the information on the card, as shown in FIG. 2, is as follows (between the two lines of asterisks):
Soluble urokinase plasminogen activator receptor levels are shown in units of ng/ml, with a range of 0.1-16.0. The analysis time is 20 min; the result is available in laboratory systems within 2 h.

Interpretation
Elevated values are observed in pathological conditions and correlate with the patient's mortality risk.
   Highly elevated values (>9) are observed in patients with multiple chronic diseases and/or serious and life-threatening conditions like severe sepsis or seriously impaired organ function. Mortality risk is highly increased.
   Moderately elevated values (about 4-9) are, for example, observed in the following conditions: Infections, cancer, COPD, cardiovascular diseases, dementia, diabetes, hepatic and renal diseases. Mortality risk and readmission risk are increased.
      Low values (<3) indicate a good prognosis.

Comments
The suPAR level should be considered in conjunction with medical history, clinical findings, and other paraclinical findings.
If the suPAR level is elevated for no obvious reason, further investigation for an unacknowledged disease may be considered.
A low suPAR level indicates a low mortality risk and a low risk of critical illness and may support a decision to discharge the subject.

### suPAR Level and Mortality Risk

Subjects below the age of 70:

| suPAR (ng/mL) | 30 days | 90 days |
|---|---|---|
| All (n = 5925) | 1.4% | 2.5% |
| 0-3 (n = 3852) | 0.2% | 0.5% |
| 3-6 (n = 1661) | 1.7% | 3.4% |
| 6-9 (n = 287) | 7.3% | 11.1% |
| >9 (n = 169) | 16.6% | 23.1% |

Subjects above the age of 70:

| suPAR (ng/mL) | 30 days | 90 days |
|---|---|---|
| All (n = 3666) | 8.8% | 15.3% |
| 0-3 (n = 750) | 2.3% | 3.5% |
| 3-6 (n = 1970) | 5.3% | 10.9% |
| 6-9 (n = 567) | 16.6% | 28.1% |
| >9 (n = 379) | 27.7% | 43.0% |

Source: The emergency departments at Hvidovre Hospital and HiHerod Hospital, Denmark n=9591.

To assess the quality of the data, and whether the physicians received and considered the suPAR level in the initial evaluation of subjects, a questionnaire was sent to 200 randomly selected physicians at the participating hospitals, asking:
Did you see the suPAR level of your subject?
Did you feel informed in the prognostic ability of suPAR?
How often did you include suPAR in your combined assessment of your subject?
How often did the suPAR level influence your clinical decision?
How often were you surprised by a high suPAR level?
How often were you surprised by a low suPAR level?

### Data Collection

Results of blood sample analyses including suPAR level were obtained from the LABKA II database. Using the unique Danish central person registration number (CPR-number), demographic data and mortality were obtained from the Central Civil Registry where all residents in Denmark are registered. Data on admissions, discharges, and diagnoses were obtained from the National Patient Registry (NPR). NPR contains information coded according to the International Statistical Classification of Disease, 10th revision (ICD-10) on primary diagnosis of discharge (A-diagnosis) and comorbidity (B-diagnoses). Laboratory values were obtained through LABKA (the clinical laboratory information system research database in Northern and Central Denmark; Grann et al (2011) Clin. Epidemiol. 3, 133-138). In the data analysis, the suPAR level from the index admission was linked with the data above to examine the primary and secondary outcomes.

### Statistical Analysis

Patients admitted in each intervention or control cycle were followed as a single cohort and data were analyzed as randomized. The two groups were assessed for comparability of the following variables: age, sex, and Charlson score. Differences in mean age of more than 5 years and/or an absolute Charlson Comorbidity Index score of 2 or more were adjusted for in the final analysis. Patient data were analyzed according to the arm of the trial to which the patient was admitted during index admission, according to the randomization scheme (Table 1) corresponding to the intention-to-treat principle. A weighted Cox model was used to compare mortality at 10 months after inclusion of the last subject. Subjects were censored if their first readmission was in the opposite group to their index admission. As this censoring is likely to be dependent censoring (a readmission is rarely a positive prognostic signal), we employed Inverse Probability of Censoring Weighting (IPCW) where subjects readmitted to their own treatment group were up-weighted to compensate. We employed stabilized weights such that the reweighted sample had the same implied sample size throughout follow-up. Due to the design, time since index admission was the only covariate that needs to be included in the weights. Reweighing was done for every two weeks of follow-up. We did not censor nor reweight for 2nd or later readmissions, since the weights would become highly unstable and it was not likely that the presence or absence of an initial suPAR measurement would be important for clinical decisions at this stage. Furthermore, a traditional intention-to-treat analysis was performed. Notable difference between the results of the two analysis strategies were considered critically. Kaplan-Meier plots were used to illustrate survival. Unpaired T-test was used to compare length of stay. P<0.05 was considered significant. Subgroup analysis of the following groups was performed: subjects aged 65 years and above, and patients discharged with diagnoses of surgical conditions, cancer, infections, and cardiovascular disease.

At follow-up (10 months after inclusion of last patient) the following data was collected from the central Danish Patient Registry:
Contacts with the healthcare system (including all historical contacts)
Information regarding admissions (date, time and place of admittance and discharge)
Diagnoses (historical and in relation to index admission).
Date of death or emigration

Diagnoses obtained from the national patient registry were coded with the ICD-10 system. The original chapters were used to group patients according to diagnoses. Primary diagnosis was used with construction subgroups, and both primary and secondary diagnoses will be used to calculate the Charlson score. The following will define the subgroups: Cancer: Chapter II: Neoplasms (COO-D48). Cardiovascular disease: Chapter IX (100-199). Infections: Chapter I: A00-699+J00-J22++N10-N11+N30-N31. Neurological disease: Chapter VI(G00-G99). Surgical conditions: Presence of surgical procedure code divided into different specialities (general, orthopedic, other).

### EXAMPLE 4

The Negative Predictive Value of suPAR Aids in Discharge Decisions

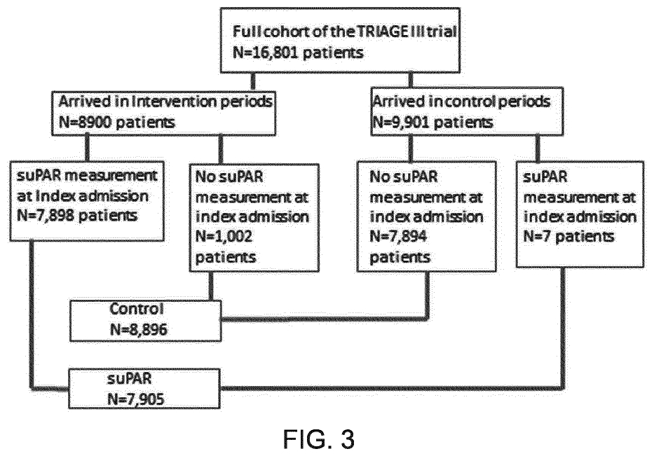

Background: The TRIAGE 111-trial is a cross-over, cluster-randomized, parallel-group, prospective, interventional trial, with the hospitals as units of randomization and the patients as the units of analysis. The trial design has been published previously (Sandø A, Schultz M, Eugen-Olsen J, et al (2016) “Introduction of a prognostic biomarker to strengthen risk stratification of acutely admitted patients: rationale and design of the TRIAGE III cluster randomized interventional trial” Scand J Trauma Resusc Emerg Med. 24(1):100. doi:10.1186/s13049-016-0290-8). We conducted the TRIAGE III-trial at the EDs of two large hospitals: Bispebjerg University Hospital and Herlev University Hospital, both located in the Capital Region of Denmark and with 70,000 and 85,000 annual admissions, respectively. By using cluster design and designating hospitals as the units of randomization, we ensured that unselected patients with different chronic- and acute diseases were included in both groups as well as a consecutive and full inclusion rate. The trial had five months of inclusion from Jan. 11, 2016 and ended as planned on Jun. 6, 2016 with a subsequent 10-month follow-up concluded on Apr. 6, 2017. The patients included are shown in FIG. 3.

Aim of study: To determine whether providing the doctors and nurses in the ED with the patient suPAR value can affect the decision of “admit or discharge” and whether providing suPAR can lead to shorter hospital length of stay.

### Methods:

suPAR levels were measured using the CE/IVD approved suPARnostic quick triage test and reader (ViroGates NS, Denmark). Data were acquired from the Danish National Patient Registry (NPR) and the Civil Registration System (CRS) at the end of follow-up (10 months after the last patient were included). All patient contacts are registered in the NPR and vital status is registered in the CRS. Data on blood tests, including plasma suPAR level, was extracted from the electronical hospital database “LABKA”. For inclusion in the trial, patients were required to have a contact in the NPR within six hours of registered blood tests in LABKA within the inclusion period and an age ≥16 years. Admissions at the pediatric, obstetric and gynaecological departments were not included. The index admission was defined as the first admission in the trial inclusion-period.

Analysis included all patients participating in the TRIAGE III trial and compared those who had a suPAR measurement (N=7,905) with those who did not (N=8,896). Differences were compared using student's T- and Wilcoxon tests. P<0.05 was considered statistically significant. Statistics were carried out using R version 1.0.136 (The R Foundation for Statistical Computing).

### Outcomes

The endpoints for the negative predictive value of suPAR were:
(I) Short admissions (<24 h) to the ED. Is there a difference in the number of patients discharged from hospital (stay shorter than 24 hours from Index) when comparing those patients who had their suPAR measured compared to those who did not?
(II) Length of stay. Is there a difference in the length of hospital stay of patients when comparing those patients who had their suPAR measured compared to those who did not?

Results: During the study, 16801 patients were included. Mean age was 60 years (SD 20) and 47.8% were men. 7905 patients had a suPAR measurement at admission and 8896 patients did not have suPAR measured (controls) (FIG. 3).

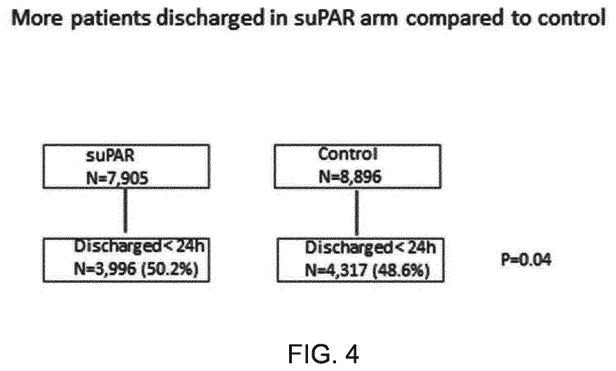

With regard to endpoint I, patients who had a suPAR measurement were significantly more often discharged within 24 hours compared to those without suPAR measurement (50.2% (3,966 patients) vs. 48.6% (4,317 patients), absolute difference: 1.6% (95% CI 0.08-3.12); P=0.039) (FIG. 4).

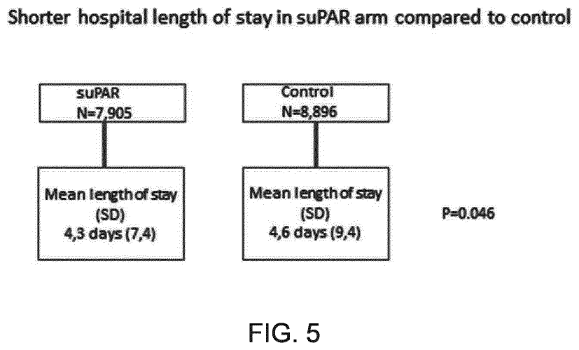

With regard to endpoint II, patients with a suPAR measurement had a 6.5 hour shorter length of hospital stay compared to patients without suPAR measurement (4.31 days (7.35) vs. 4.58 days (9.37), difference: 0.27 days (95% CI 0.01-0.53), P=0.043) (FIG. 5).

Mortality in Patients Discharged within 24 Hours

All-cause mortality within 30 days among early discharged patients occurred in 52 patients (1.3%) in the suPAR group and in 77 patients (1.8%) in the control group. The unadjusted Cox model found a trend towards lower mortality in the suPAR group compared to control: Hazard ratio (HR), 0.73; 95% confidence interval (CI) 0.52 to 1.04; P=0.084.

During the median 12-months of follow-up, 225 (5.7%) of the patients died, which was less than among early discharged patients in the control arm where 256 (6.7%) died during follow-up (P=0.05). In patients that were discharged within 24 hours, the AUC for predicting 30-day mortality was 0.92 (95% CI: 0.90-0.95)

Readmissions in Patients Discharged within 24 Hours

With regard to 30-day readmission, 336 (8.5%) patients in the suPAR group were readmitted, while 331 (7.7%) patients in the control group were readmitted, P=0.18. For 90-day readmission, 490 patients (12.4%) vs. 552 patients (12.8%) were readmitted in the suPAR group and control group, respectively (P=0.57).

Discussion: The study showed that knowledge of patient's suPAR level at the Emergency Department led to earlier discharged patients and overall shorter length of stay. Even though more patients were discharged in the suPAR group compared with controls, there was no difference with regard to readmissions or mortality. Thus, early discharge based on suPAR is safe and feasible. Improving patient flow and earlier discharge of patients where admission might not be necessary will benefit both patients in need of hospital treatment and low-risk patients who can be discharged without being exposed to the risks of hospitalization, such as in-hospital infections, loss of muscle mass and loss of personal income if the patient is working. For the hospital, the shorter admission observed in patients that had suPAR measured at admission (6 hours shorter in the suPAR arm), leads to economic savings.

The fact that the AUC of suPAR became very high among those early discharged shows that the doctors used the positive predictive value of suPAR and kept patients more than 24 hours in hospital if suPAR was elevated. The high AUC of 0.92 thus reflects that those early discharged were the low risk patients and those who were sent home to die (e.g. to hospice or retirement home).

### EXAMPLE 5

Positive Predictive Value of suPAR

Background: suPAR has previously been shown to be a strong predictor of outcome in retrospective studies. However, it was unknown whether giving the doctors information on the suPAR level could alter the outcome/change the prognosis. In the TRIAGE III Intervention study, suPAR was measured at time of admission using the suPARnostic Quick Test in 7,905 patients. Comparison is made to the 8896 patients in the control arm (without suPAR measurement) (FIG. 3).

Methods: suPAR levels were measured using the CE/IVD approved suPARnostic quick triage test and reader (ViroGates NS, Denmark). The discriminative ability of suPAR with regard to mortality at one and ten months was assessed by using area under the curve (AUC) for receiver operating characteristics (ROC).

P<0.05 was considered statistically significant. Statistics were performed in R version 1.0.136 (The R Foundation for Statistical Computing) and figures were created with Graphpad Prism, version 7.02.

### Results:

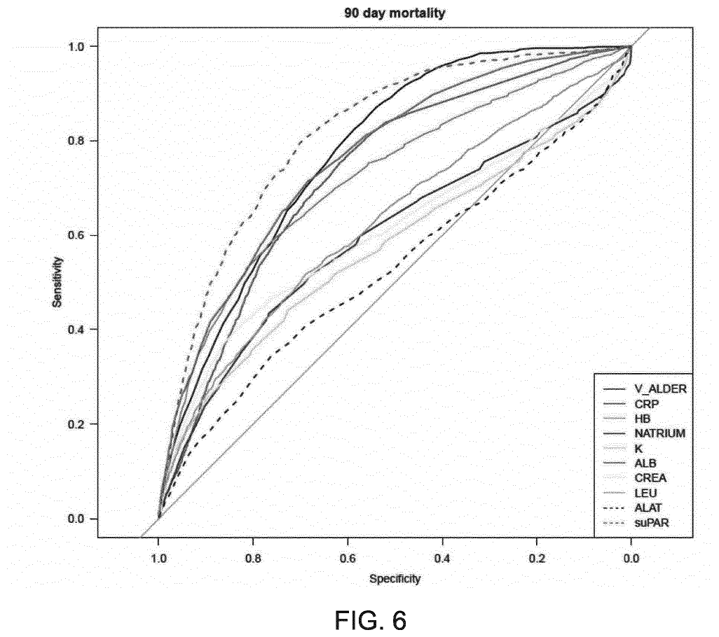

suPAR and mortality. The median suPAR level of patients who survived was significantly lower than the suPAR level of patients who died during follow-up, both at 30 days (4.0 ng/ml (IQR 2.9-5.7) vs. 8.3 ng/ml (IQR 5.9-11.7), p<0.001) and 10 months (3.8 ng/ml (IQR 2.8-5.3) vs. 6.9 ng/ml (IQR 5.1-10.1), p<0.001). SuPAR had a high prognostic power for predicting 30-days and 10-months mortality (AUCs: 30 days: 0.83 (95% CI: 0.81-0.84); 10 months: 0.80 (95% CI: 0.79-0.82). In comparison with age and routine biomarkers, suPAR had superior prognostic power regarding mortality at all follow-up times (Table 2: AUC for suPAR and other routine biomarkers and age) (FIG. 6, ROC curve analysis for single markers and their ability to predict 30-day mortality; the dashed line to the left of the figure is the level of suPAR).

TABLE 2
Area Under the Curve (AUC)for the
routine measured biomarkers and age

| | | | Mortality |
|---|---|---|---|
| | Mortality 30 days | Mortality 90 days | All follow-up |
| Age | 0.777 | 0.774 | 0.781 |
| C-reactive | 0.738 | 0.729 | 0.702 |
| protein | | | |
| Hemoglobin | 0.701 | 0.721 | 0.729 |
| Sodium | 0.582 | 0.597 | 0.604 |
| Potassium | 0.578 | 0.574 | 0.564 |
| Albumin | 0.777 | 0.763 | 0.732 |
| Creatinine | 0.622 | 0.607 | 0.604 |
| Leucocytes | 0.654 | 0.627 | 0.580 |
| ALAT¹ | 0.511 | 0.530 | 0.550 |
| suPAR | 0.835 | 0.815 | 0.802 |

Adding suPAR to Algorithm Significantly Improves Outcome Prediction ¹ Alanine aminotransferase

To determine whether suPAR provides an additional and independent value to a combined model of all predictive routine markers, two models were made: one without suPAR but containing all the variables found significant in Table 2, and another model including these variables and suPAR.

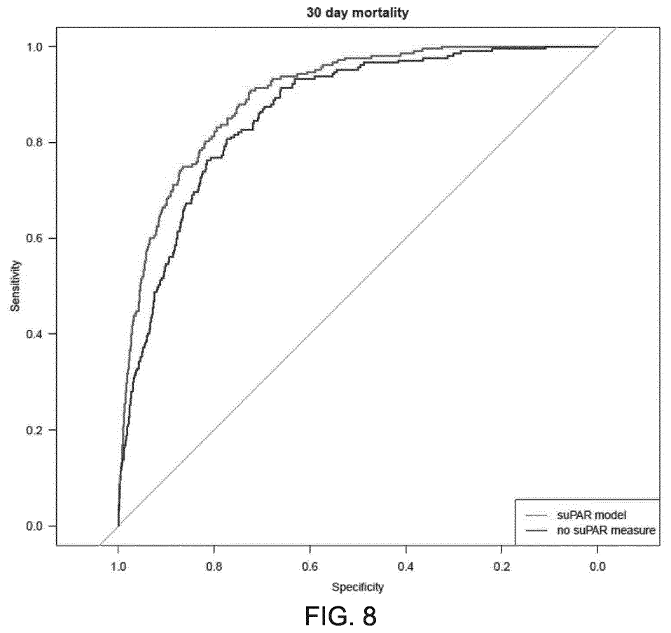

For the prediction of 30-day mortality, the first model (without suPAR) provides an AUC of 0.860 (95% CI 0.84-0.86). Addition of suPAR significantly improved this model, AUC 0.896 (95% CI 0.88-0.90), p=0.007. The increase in sensitivity and specificity can be seen in FIG. 8.

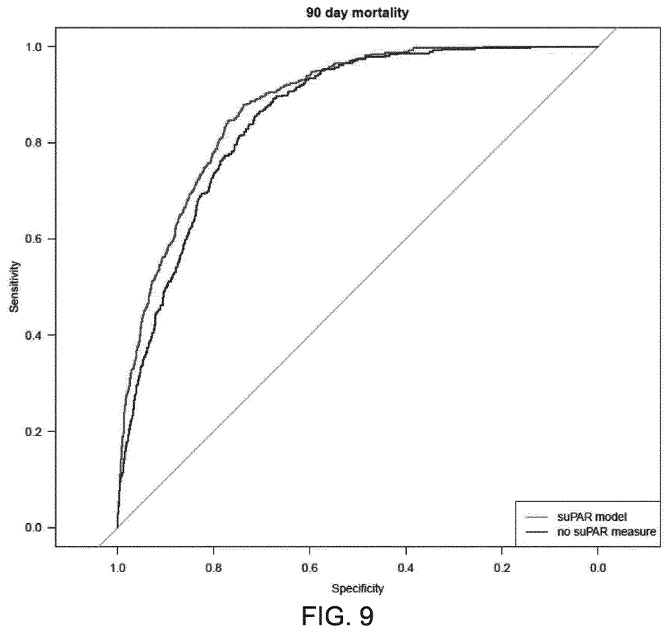

Similarly, for the determination of 90-day mortality, the model without suPAR provided an AUC of 0.854 (95% CI: 0.84-0.85). When including suPAR, the model significantly improved to an AUC of 0.878 (95% CI: 0.86-0.88), p=0.001 (FIG. 9).

Measuring suPAR at Admission and Difference in Mortality Between Patients with or without suPAR Measurement

With regard to mortality in the suPAR intervention arm versus the control, we observed a mortality rate of 13.9% in the intervention arm compared to 14.3% in the control arm corresponding to 36 fewer mortalities in the intervention arm. The difference in mortality between the suPAR Intervention arm and control arm was strongly observed at Bispebjerg Hospital, Copenhagen, Denmark. At Bispebjerg Hospital, 3451 patients were included in the suPAR intervention arm and 3569 in the control arm. During follow-up, 427 patients died in the suPAR intervention arm (12.4%) which was a significant lower mortality than was observed in the control arm (515 died (14.4%), p<0.05.

Discussion: In this study, it is shown that suPAR is superior to other biomarkers with regard to outcome prediction compared with other investigated biomarkers, including a combined model of commonly used routine blood tests, in predicting short-term mortality. It is of interest that suPAR, in contrast to other biomarkers, is stronger than age in prediction of outcome. Also, adding suPAR to an algorithm of all the routine biomarkers significantly improved the prediction of both 30- and 90-day mortality. With regard to prevention of mortality, less mortality was observed in the intervention arm compared with the control arm. The effect of informing the doctors of suPAR level was of most value in patients with well-functioning clinical signs, e.g. in those triaged in the low risk category or having a low Early warning score (EWS or NEWS) where a severe disease, if present, is not recognised without the suPAR measurement.

The prognostic abilities of suPAR have been studied retrospectively before, and the biomarker has been shown to be associated with risk of mortality and adverse events. However, previous studies have not investigated the clinical impact of interventions on patients when giving the doctors “real time” information on the suPAR level while the patient was present in the ED. Hence, it was until now unknown whether knowledge of suPAR while the patient is present can change the outcome of the patient.

This study shows for the first time that knowledge of suPAR led to more early discharges in the Intervention arm compared with control. With regard to mortality in those early discharged, fewer patients died in the intervention arm compared with control, demonstrating that both the negative and positive predictive value of providing “real time” suPAR levels to the doctors and nurses aids in better admission and discharge decisions in the Emergency Departments.

1. A method of applying risk stratification to a human subject who has been admitted to, or presents at, a hospital emergency department (ED), the method comprising measuring the soluble urokinase type plasminogen activator (suPAR) level in a sample obtained from the subject and comparing it with a reference suPAR value.

2. A method according to claim 1 comprising determining the morbidity of the subject.

3. A method according to claim 1 or 2 comprising determining the risk of in-hospital death or death within 28 days, 90 days, 6 months, 10 months or 2 months of the subject.

4. A method according to any of the preceding claims comprising determining the need to admit the subject into the hospital

5. A method according to any of the preceding claims comprising determining the ability to discharge the subject from the hospital or not to admit the subject into the hospital.

6. A method according to any of the preceding claims wherein the sample is blood, blood serum, blood plasma, cerebrospinal fluid or urine.

7. A method according to any of the preceding claims wherein the risk stratification additionally comprises measuring and/or processing one or more of: the subject's sex, age, medical history, haemoglobin level, C Reactive Protein level, creatinine level, leucocyte count, sodium level, potassium level, adrenomedullin level, albumin level, D-dimer level, troponin level (HEART Score); recording clinical symptoms and signs such as physiological parameters, such as pulse, cognition, blood pressure, temperature and respiratory rate; the output of a risk algorithm such as Early warning score and similar and locally adapted variables thereof (e.g. Decision-tree early warning score (DTEWS) or National Early Warning Score (NEWS), Acute Physiology and Chronic Health Evaluation (APACHE), Glasgow coma scale, electrocardiogram, age, risk factors, quick Sepsis Related Organ Failure Assessment (qSOFA), or the Model for Endstage Liver Disease (MELD), based on bilirubin, INR (international normalized ratio), and creatinine); the American Society of Anesthesiologists (ASA) classification; the Physiologic and Operative Severity Score for the enUmeration of Mortality and Morbidity (POSSUM) score; or other risk scores for outcome prediction of acute hospitalized patients, such as the GRACE ACS Risk and Mortality Calculator, the Thrombolysis in Myocardial Infarction risk score (TIMI RS), Platelet glycoprotein IIb/IIIa in Unstable angina: Receptor Suppression Using Integrilin Therapy risk score (PURSUIT RS), and Global Registry of Acute Cardiac Events risk score (GRACE RS) for in-hospital and 1 year mortality across the broad spectrum of non-ST-elevation acute coronary syndromes (ACS).

8. A method according to any of the preceding claims wherein the reference suPAR value is a plasma level of between 0 and 16 ng/ml.

9. A method according to claim 8 wherein a plasma suPAR level of higher than 4 ng/ml in the subject is a factor indicating that a subject should be admitted as a patient, or kept in as a patient, even if other components of the risk stratification procedure are factors indicating that the subject need not be admitted or can be discharged.

10. A method according to claim 8 or 9 wherein a plasma suPAR level of higher than 6 ng/ml, especially higher than 9 ng/ml, in the subject is a strong factor indicating that a subject should be admitted as a patient, or kept in as a patient, even if other components of the risk stratification procedure are factors indicating that the subject need not be admitted or can be discharged.

11. A method according to any of claims 8 to 10 wherein a plasma suPAR level of lower than 4 ng/ml, especially lower than 3 ng/ml, is a factor indicating that a subject need not be admitted as a patient, or can be discharged from the hospital.

12. A method according to any of the preceding claims wherein the subject's suPAR level is measured within 6 hours of the subject's arrival at the hospital emergency department.

13. Apparatus for applying risk stratification to a human subject who has been admitted to, or presents at, a hospital emergency department (ED), the apparatus comprising:
means to accommodate a sample obtained from the subject,
a detector configured to measure the level of soluble urokinase type plasminogen activator (suPAR) in the sample,
a processing module to compare the level of suPAR with a reference suPAR value, and
means to output a risk stratification.

14. Apparatus according to claim 13 wherein the means to output the risk stratification is a visual display or a printout.

15. Apparatus according to claim 13 or 14 wherein, in order to output the risk stratification, the apparatus additionally processes one or more of measuring and/or processing one or more of: the subject's sex, age, medical history, haemoglobin level, C Reactive Protein level, creatinine level, leucocyte count, sodium level, potassium level, adrenomedullin level, albumin level, D-dimer level, troponin level (HEART Score); recording clinical symptoms and signs such as physiological parameters, such as pulse, cognition, blood pressure, temperature and respiratory rate; the output of a risk algorithm such as Early warning score and similar and locally adapted variables thereof (e.g. Decision-tree early warning score (DTEWS) or National Early Warning Score (NEWS), Acute Physiology and Chronic Health Evaluation (APACHE), Glasgow coma scale, electrocardiogram, age, risk factors, quick Sepsis Related Organ Failure Assessment (qSOFA), or the Model for Endstage Liver Disease (MELD), based on bilirubin, INR (international normalized ratio), and creatinine); the American Society of Anesthesiologists (ASA) classification; the Physiologic and Operative Severity Score for the enUmeration of Mortality and Morbidity (POSSUM) score; or other risk scores for outcome prediction of acute hospitalized patients, such as the GRACE ACS Risk and Mortality Calculator, the Thrombolysis in Myocardial Infarction risk score (TIMI RS), Platelet glycoprotein IIb/IIIa in Unstable angina: Receptor Suppression Using Integrilin Therapy risk score (PURSUIT RS), and Global Registry of Acute Cardiac Events risk score (GRACE RS) for in-hospital and 1 year mortality across the broad spectrum of non-ST-elevation acute coronary syndromes (ACS).
